(19) United States
(12) Patent Application Publication
(10) Pub. No.: US 2014/0017689 A1
(43) Pub. Date: Jan. 16, 2014

(54) METHOD FOR DETECTING NUCLEIC ACIDS

(71) Applicant: Abacus Diagnostica Oy, Turku (FI)

(72) Inventors: Piia VON LODE, Paattinen (FI); Anniina WESTER, Turku (FI); Timo LOVGREN, Kirjala (FI); Tero SOUKKA, Turku (FI)

(73) Assignee: ABACUS DIAGNOSTICA OY, Turku (FI)

(21) Appl. No.: 14/016,952

(22) Filed: Sep. 3, 2013

Related U.S. Application Data
(63) Continuation of application No. 13/266,709, filed on Oct. 27, 2011, now abandoned.
(60) Provisional application No. 61/177,547, filed on May 12, 2009.

(30) Foreign Application Priority Data
May 12, 2009 (FI) 20095531
May 12, 2009 (FI) 20095532

(51) Int. Cl. C12Q 1/68

(52) U.S. Cl. CPC C12Q 1/6818
USPC 435/6.11; 436/501

(57) ABSTRACT

Method for detecting nucleic acids which employs a double-stranded oligonucleotide probe containing i) a first probe including a first label moiety, and ii) a second probe partially complementary with the first probe and including a second label moiety capable of interacting with the first moiety when brought in close proximity with each other, the second moiety being a quencher or acceptor of emission of the first moiety. The first or second probe includes a sequence complementary to that of a target nucleotide, and the second or first probe, respectively, includes a sequence complementary to a complement of the target nucleotide sequence of the nucleic acid to be detected. Oligonucleotides for determining Chlamydia trachomatis are also disclosed.

### FIELD OF THE INVENTION

This invention relates to a homogenous competitive method for detecting nucleic acids. This invention further relates to nucleic acid sequences for detecting Chlamydia trachomatis.

### BACKGROUND OF THE INVENTION

The publications and other materials used herein to illuminate the background of the invention, and in particular, cases to provide additional details respecting the practice, are incorporated by reference.

Several nucleic acid amplification techniques have become available starting from the mid-80's. The polymerase chain reaction (PCR; Saiki R K et al. Science 1985; 230:1350-4) is a wide-spread nucleic acid amplification technique that has become one of the most important tools in nucleic acid detection and diagnostics. In PCR, a specific target DNA sequence is amplified to an extent where it can be detected, for example, with the help of sequence-specific, short oligonucleotides (probes) labelled e.g. with fluorescent labels. The binding sites of the oligonucleotide probes are located between the PCR primers used to amplify the target nucleic acid. In theory, even one copy of a target nucleic acid (e.g., a specific sequence in the DNA genome of e.g. one bacterial cell) can be amplified by PCR up to a detectable level, allowing extremely sensitive detection of pathogens, cancer cells, single-nucleotide polymorphisms and other targets that can be identified by specific nucleic acid sequences. In practice, however, the sensitivity of nucleic acid detection is often limited by the selection of the methods and labels used in the detection and identification of the amplified PCR products.

Traditional methods for nucleic acid detection require heterogeneous post-PCR assay steps such as restriction enzyme analysis, agarose gel electrophoresis, or heterogeneous hybridization steps where unhybridized and hybridized probes are physically separated from each other. All such methods constitute a serious risk of cross-contamination by the amplification products. In the state-of-the art nucleic acid detection methods, however, the detection of amplification products can be performed directly from the PCR assay vessel in a homogeneous environment, i.e., without opening the reaction vessel during or after PCR. Homogeneous detection that is performed as a separate step after the amplification step has been completed is called homogeneous end-point detection. The accumulation of the target DNA can also be homogeneously monitored during PCR by use of so-called real-time PCR methods.

The homogeneous detection methods, both end-point and real-time, can further be classified into probe-using and non-probe-using formats. Both formats, however, typically utilize different kinds of fluorescent labels and fluorescence detection techniques, exemplified e.g. in U.S. Pat. No. 5,994,056, U.S. Pat. No. 5,804,375, EP 0 543 942, U.S. Pat. No. 5,928,862, U.S. Pat. No. 6,902,900, U.S. Pat. No. 6,635,427, EP 0 912 760, EP 0 745 690 and WO 2008/093002. The principle of homogeneous detection by using competitive hybridization is described e.g. in U.S. Pat. No. 5,928,862, EP 1 339 732, EP 0 861 906, Li Q et al. 2002 (Nucleic Acids Res 2002; 30:E5) and Cheng J et al. 2004 (Nucleic Acids Res 2004; 32:E61). In competitive hybridization, two complementary oligonucleotide probes, one typically labelled with a fluorophore and the other typically with a quencher (or a fluorescence acceptor), are at a suitable temperature allowed to hybridize with their respective amplified targets (i.e., the complementary strand of the one- or two-stranded product) and with each other. When the two probes are hybridized together forming a double-stranded oligonucleotide probe, the fluorescence of the label probe is substantially quenched by the close proximity of the quenching moiety of the quencher probe. When bound to the target, the label probe becomes substantially unquenched, leading to an increased level of detectable light. The more target present, the larger the portion of light-emitting label probe. There is thus competition for the label probe between the target and the quencher probe typically for the same target nucleic acid sequence, i.e. without the need for surplus probe-specific nucleotide sequence not present in the target nucleic acid, such as is required in probes containing a stem-structure for probe self-hybridization (e.g. EP 0 745 690, EP 0 728 218) and in dual-purpose oligonucleotides simultaneously used as primers and probes (WO 2009/042851 A1). A special case of competitive hybridization is, however, described in EP 1 726 664 where competition is not based on a nucleic acid sequence inherently present in the target nucleic acid but where a synthetic target sequence is incorporated in the amplification product during the amplification process. However, the use of surplus nucleotide sequence that has no intentional complementarity with a target nucleic acid sequence or its complement renders the oligonucleotide probes susceptible for undesired and unpredictable nonspecific hybridization reactions and also limits their use e.g. in multiplexed reactions and in allelic discrimination assays. As described e.g. in EP 1 339 732, EP 0 861 906, EP 1 726 664, Li et al., 2002 and Cheng et al., 2004, the competitive nature of the hybridization process in methods utilizing competitive hybridization is further enhanced by using probes with unequal lengths so that the quencher probe typically is shorter than the label probe, allowing the label probe to bind to the amplified target with higher strength and thus giving it competitive advantage. The comparative binding strength can be assessed by calculating the melting temperature, Tₘ, of the formed double-strand. In the competitive hybridization methods described in earlier publications, the Tₘ of the label probe-target-hybrid is thus typically higher than the Tₘ of the double-stranded oligonucleotide probe.

There are some shortcomings associated with the above competitive hybridization methods and the related reagent development. For example, the described methods favour the use of single-stranded targets because they typically employ a probe pair made of two complementary oligonucleotides of different lengths. As explained in EP 1 339 732, double-stranded oligonucleotide probes having strands of different lengths can spontaneously react with single-stranded target oligonucleotides by a mechanism where the short strand is displaced by the target nucleic acid to form a thermodynamically more stable duplex, producing an increase in fluorescence. However, while single-stranded target nucleic acids can be produced e.g. by using so-called asymmetric amplification techniques, the most common and efficient types of nucleic acid amplification methods produce double-stranded oligonucleotide products. Therefore, even though a longer label probe was used to induce preferred hybridization of the label probe with the amplified target as described in EP 1 339 732 and EP 0 861 906, the quencher probe competes for the amplified target nucleic acid and the label probe with the same strength. This leads into a situation where the quencher probe, typically also being present in relatively large excess compared to the label probe, continues to very strongly compete for the label probe thus decreasing the proportion of light-emitting label probe and, therefore, the sensitivity of the assay especially in the presence of low amounts of the target nucleic acid. Adjustment of the hybridization temperature to decrease the reciprocal hybridization of the label and quencher probes, on the other hand, leads to a lower quenching efficiency and thus a higher background signal level in situations where the amplified target is absent, thus again decreasing the sensitivity of the assay.

Furthermore, in the case of a double-stranded target nucleic acid, it is said e.g. in EP 1 339 732 and U.S. Pat. No. 5,928,862 that double-stranded oligonucleotide probes having complementary oligonucleotides of equal length can be employed. It is thus assumed that because the displacement mechanism of the shorter probe strand by a single-stranded target nucleic acid cannot be employed, and because two complementary target strands are present to bind both oligonucleotide probes, decreasing the length of one of the oligonucleotide probes is no longer necessary.

One solution to the problem has been provided in EP 1 911 852 where the binding of the single-stranded oligonucleotide probes to the respective target nucleic acid strands is favoured over the mutual binding of the two probe strands. This is achieved by providing, on each strand of the double-stranded probe, a nucleotide sequence designed to bind to the target nucleic acid but not to the other strand of the probe. However, the single-stranded oligonucleotide probes according to EP 1 911 852 inherently contain spacer moieties, typically consisting of a sequence of surplus nucleotides, which do not participate in the specific hybridization reactions but separate the fluorescent donor and fluorescent acceptor moieties physically from each other. The spacer moieties thus hinder efficient interaction between the fluorescent donor and acceptor moieties and also render the probes susceptible to unwanted nonspecific hybridization reactions, especially because there are no intentional counterparts present in the reaction to hybridize with the surplus nucleic acid sequence of the spacer moiety; instead, the single-stranded spacer moiety is free to spontaneously react with other nucleic acids present e.g. in the sample. Furthermore, the double-labelling of both of the single-stranded oligonucleotide probes with fluorescent donor and fluorescent acceptor moieties at the same time leads to unintentional self-quenching of the oligonucleotide probes whether in single-stranded or in hybridized form. The oligonucleotide probes according to EP 1 911 852 do not therefore provide means to increase the sensitivity of detecting low amounts of target nucleic acids over existing methods.

Chlamydia trachomatis (C. trachomatis) is an obligate intracellular Gram-negative bacterium and the causative agent of a very common sexually transmitted disease. Due to the high morbidity and multiple adverse effects of a C. trachomatis infection, rapid and specific diagnostic tests are of high importance. Diagnosis based on selective culture of the organism has been the “golden standard”, but is rapidly being replaced by direct detection of organism-specific nucleic acid sequence or sequences mainly due to two reasons: firstly, cell culturing is time-consuming and C. trachomatis is difficult to grow by culture; and secondly, especially PCR-based nucleic acid amplification methods have proven extremely useful in detecting microbial pathogens in clinical settings and the testing volumes are increasing rapidly. Selection of the target sequence or sequences for amplification (i.e., amplicons) determines the specificity and, in part, the sensitivity of the C. trachomatis assay. Most commonly the target sequence is selected within the cryptic plasmid (extra-chromosomal DNA) of C. trachomatis. The cryptic plasmid is about 7500 by of length and is highly conserved across isolates (Comanducci M et al. Plasmid 1990;23:149-54). Furthermore, the cryptic plasmid is present in multiple copies (5-10) per organism. Accordingly, numerous suitable target sequences specific for the C. trachomatis cryptic plasmid have been published, recently e.g. in U.S. Pat. No. 2008/299567, WO 2008/097082, WO 2007/056398, WO 2007/137650 and U.S. Pat. No. 2007/065837.

The present invention provides a new target sequence in the cryptic plasmid that is useful for the specific amplification and detection of C. trachomatis.

### OBJECT AND SUMMARY OF THE INVENTION

One object of the present invention is to provide a method for detecting nucleic acids.

Another object of the invention is to provide nucleic acid sequences for use as primers in a nucleic acid amplification assay determining Chlamydia trachomatis.

The present invention provides a method for detecting nucleic acids wherein a double-stranded oligonucleotide probe comprising
i) a first single-stranded oligonucleotide probe comprising at least one first label moiety capable of emitting a measurable signal, and
ii) a second single-stranded oligonucleotide probe being partially complementary, i.e. an essential part of the probe being essentially complementary, with the first single-stranded oligonucleotide probe and comprising at least one second label moiety capable of interacting with said first label moiety when brought in close proximity with each other, the second label moiety being a quencher or acceptor of emission of the first label moiety;

wherein said first or second oligonucleotide probe comprises a sequence being essentially complementary to that of a target nucleotide sequence, and said second or first oligonucleotide probe, respectively, comprises a sequence being essentially complementary to a complement of said target nucleotide sequence of said nucleic acid to be detected; and
wherein said first and said second label moieties are attached to said first and second oligonucleotide probes respectively in a manner wherein the distance between said first and second label moieties of said double-stranded oligonucleotide probe is not more than 7 base pairs, preferably not more than 4 base pairs, more preferably not more than 2 base pairs apart and most preferably said first and second label moieties are attached to the same base pair of the said double stranded oligonucleotide probe;

is employed; and said method being characterized in that

a) the complementary sequences of said double-stranded oligonucleotide probe, i.e. the sequences of the first and second oligonucleotide probe being essentially complementary to each other, being shorter than the full sequence of either of said first and second single-stranded oligonucleotide probes;

b) said first and second oligonucleotide probes having a higher Tₘ when hybridized with said target nucleotide sequence compared to the Tₘ of said double-stranded oligonucleotide probe; and

c) the intensity of the signal of said first label when said first oligonucleotide probe is not hybridized to said second oligonucleotide probe being higher or lower, preferentially higher, than the intensity of the signal of said first label when said first oligonucleotide probe is hybridized to said second oligonucleotide probe.

The present invention further provides single-stranded oligonucleotides having at least 90% identity with SEQ ID NOS: 2 and 3 for use as primers in a nucleic acid amplification assay determining Chlamydia trachomatis.

### BRIEF DESCRIPTION OF THE DRAWINGS

FIG. 1 illustrates the probes employed in the method of the present invention in relation to the target nucleic acid.

FIG. 2 illustrates the results of Example 1 illustrating detection of Neisseria gonorrhoeae employing prior art methods [equal-length complementary probes (x), unequal-length complementary probes (∘)] in comparison to the method of the present invention [partially complementary Quencher probe with a 2-bp single-stranded extension (▴), partially complementary Quencher probe with a 3-bp single-strand extension ()].

FIG. 3 illustrates the results of the experiments of Example 2. FIG. 3 shows end-point detection of C. trachomatis in homogeneous PCR using unequal length complementary probes (∘) and partially complementary probes of the current invention ().

FIG. 4 illustrates homogeneous real-time monitoring of the accumulation of MRSA-specific DNA in PCR using unequal-length complementary probes (open symbols) and partially complementary probes of the current invention as disclosed in Example 3. Sample dilutions used: 1:100 (⋄/♦), 1:1 000 (∘/), 1:10 000 (□/▪), 1:100 000 (Δ/▴) and 1:1 000 000 ( [symbol] /x). Negative reactions are indicated with dotted (unequal-length complementary probes) and solid (partially complementary probes) lines with no symbols.

### DETAILED DESCRIPTION OF THE INVENTION

The present invention relates to a method for detecting nucleic acid amplification in a homogeneous assay format utilizing competitive hybridization. The method is based on using a pair of two partially complementary oligonucleotide probes, one of which is labeled with a fluorescent reporter molecule (Label probe) and the other with a quencher molecule (Quencher probe) that substantially quenches the fluorescence from the reporter whenever the two probes are in close proximity, i.e. hybridized with each other. The Quencher probe could be similarly labeled with an acceptor molecule for the purpose of absorbing the signal emitted by the label of the Label probe when in close proximity, and typically but not necessarily transforming it to a different detectable signal. The type of label molecules and interaction mechanism used are freely selectable depending on the detection technology employed. An important feature of the invention is that both the Label probe and the Quencher probe are only partially complementary to each other, so that both of the probes contain additional nucleotides the counterparts of which are typically present only in the respective strand of the target nucleic acid. Consequently, both the probes more preferentially hybridize with the target nucleic acid that accumulates during e.g. PCR amplification rather than hybridize with each other. In other words, both strands of the target nucleic acid compete for the probes more strongly than the probes compete for each other, leading to an increased sensitivity of detection as compared with other competitive hybridization methods.

The current invention demonstrates that it is of high significance to incorporate single-stranded segments in not only one but in both of the probes. It is important to notice that this is not for the purpose of easier displacement of one of the probes, but rather to strengthen the competition of the target nucleic acid for the probes by increasing the Tₘ of the probe-target (comprising both Label probe-target and Quencher probe-target) complexes, as compared with the Tₘ of the double-stranded oligonucleotide probe. This kind of probe design increases detection sensitivity by two ways: firstly, the Label probe more likely hybridizes with the target nucleic acid than it hybridizes with the Quencher probe, allowing direct generation of more fluorescence due to the competitive advantage of the Label probe-target hybrid. Secondly, although there is no direct effect on fluorescence whether the Quencher probe is free in solution or bound to the target, there is an indirect mechanism that further increases the level of fluorescence when the target nucleic acid is present: the more Quencher probe hybridized with the target, the less there is to hybridize with the Label probe; in other words, a significant portion of free Quencher probe can be pulled out of the reaction to further decrease the competition of the Quencher probe for the Label probe. It must be noted that a free (single-stranded) Label probe is similarly unquenched as a Label probe bound to the target oligonucleotide. The detection sensitivity can thus be enhanced by two different mechanisms, as also demonstrated in Example 1. The detection sensitivity of competitive probes is thus determined by the competitive characteristics of the probes and, as presented in the current invention, these characteristics can be easily modified to obtain higher detection sensitivity especially of low amounts of target nucleic acids.

Specific oligonucleotides for the amplification and detection of Chlamydia trachomatis are presented as well.

The present invention relates to a homogeneous method for the detection of a target nucleic acid using competitive hybridization probes. The method can be similarly applied as an internal part of a nucleic acid amplification method, such as real-time monitoring of the accumulation of target in PCR, or as a separate step after the amplification step has been completed, such as in post-PCR end-point detection.

The present invention provides a method for increasing the detection sensitivity of competitive probes in detecting the presence of specific target nucleic acids. A typical method according to the invention comprises
a) providing a mixture of a sample potentially including the specific target nucleic acid and a double-stranded oligonucleotide probe consisting of a Label probe labelled e.g. with a fluorophore and a Quencher probe labelled e.g. with a quencher, both of the probes being capable of hybridizing with each other and with the respective strand of the target nucleic acid;
b) designing both the Label probe and the Quencher probe so that in the presence of the target nucleic acid the probes are preferentially bound to the target nucleic acid rather than to the complementary probe, achieved by dimensioning the probes so that the Tₘ of the complementary sequence of the two probes (Tₘ1) is several degrees of Celsius lower compared with the Tₘ of the complementary sequence of the Label probe-target oligonucleotide duplex (Tₘ2) and the Tₘ of the complementary sequence of the Quencher probe-target oligonucleotide duplex (Tₘ3);
c) exposing the mixture to conditions where the probes and the target nucleic acid can assume the thermodynamically more favoured complexes, for example by denaturing all oligonucleotides in the mixture by exposing them to a high temperature, and then allowing them to hybridize with each other by lowering the temperature of the reaction, for example performed as an internal step of a PCR amplification reaction (real-time monitoring of the accumulation of target), or as a separate step after the amplification reaction has been completed (end-point detection);
d) measuring the signal, e.g. fluorescence, at least once after allowing the oligonucleotides to hybridize with each other; in certain embodiments the signal can also be measured before this hybridization step to allow comparing the intensity of signal in the different measurement conditions; and
e) determining whether the target nucleic acid is present in the mixture or not, for example based on comparing the intensity of signal to a pre-determined cut-off value, or quantifying the amount target nucleic acid in the original sample by interpolating the obtained signal or result calculated based on the signal in a distinctive standard curve.

Furthermore, a nucleotide sequence (SEQ ID NO: 1) is provided that allows the design of oligonucleotides specific for C. trachomatis. The oligonucleotides can be used in a method to specifically amplify and/or detect C. trachomatis in a sample, the method typically comprising the steps of:
(a) forming a reaction mixture comprising nucleic acid amplification reagents, at least one pair of oligonucleotide primers and a test sample that is analyzed for the presence of C. trachomatis-specific DNA. The mixture preferably also contains a pair of oligonucleotide probes (Label probe and Quencher probe) that allow producing a detectable signal upon amplification of the target sequence when present in the reaction;
(b) subjecting the mixture to amplification conditions to generate multiple copies of the target nucleic acid sequence and/or its complement;
(c) allowing the oligonucleotide probes present in the reaction to hybridize to the target nucleic acid sequence and/or its complement so as to form probe-target hybrids when the target is present; and
(d) measuring the signal of the reaction at least once to determine the presence or absence of the target sequence in the sample. It is also possible to quantify the amount target nucleic acid in the sample e.g. by interpolating the obtained result in a distinctive standard curve or by some other means.

One skilled in the art will understand that the method for detecting C. trachomatis in the sample may not contain all steps as outlined above, or may contain additional steps that are not included in the description above which is of general nature.

Definitions

The term “oligonucleotide” as used herein includes linear oligomers of natural or non-natural monomers or linkages, including but not being limited to deoxyribonucleosides and ribonucleosides, forming a nucleic acid. The monomers of a nucleic acid strand are most often referred to as bases, because two nucleotides on opposite nucleic acid strands are connected via hydrogen bonds forming a base pair (bp). Whenever an oligonucleotide is represented by a sequence of letters, such as “TATGACCA”, it shall be understood that the nucleotides are in 5′→3′ order from left to right. The four bases found in DNA and referred to in the current invention are adenine (A), cytosine (C), guanine (G) and thymine (T). The length of an oligonucleotide is measured as the number of consecutive bases; the minimum difference between two unequal-length oligonucleotides is thus one base. Oligonucleotides are generally conceived as relatively short, often chemically synthesized nucleic acid polymers with a length typically between 5 and 200 bases. Nucleic acid segments longer than this are often referred to as “polynucleotides”. The term “nucleic acid” is the most comprehensive term in this respect and refers to a macromolecule of any size composed of one (single-stranded nucleic acid) or two (double-stranded nucleic acid) polymeric chains of nucleotides. The most common nucleic acids are deoxyribonucleic acid (DNA) and ribonucleic acid (RNA). Nucleic acid molecules carry the genetic information of all living organisms. The term “sequence” as used herein refers to the type and order of bases in the nucleic acid.

Oligonucleotides can readily bind to their respective complementary oligonucleotide, and they are often used as primers and/or probes to allow amplification, detection and/or quantifying target nucleic acids. Examples of procedures that use oligonucleotides include PCR, DNA microarrays, Southern blots, fluorescent in situ hybridization (FISH), and the synthesis of artificial genes.

The term “primer” as used in the present invention means an oligonucleotide that is employed in a nucleic acid amplification method such as PCR to allow a polymerase to extend the oligonucleotide and replicate the complementary strand. Specifically, a primer serves as a starting point for DNA replication, and the polymerase starts replication at the 3′-end of the primer. A primer typically comprises 10 to 40 bases, but can also be shorter or longer depending on the application in which it is used.

The term “probe” as used in the present invention means an oligonucleotide that is employed to detect and/or quantify the target nucleotide in a sample, typically during or after a nucleic acid amplification reaction. A probe contains a nucleotide sequence that is complementary to a sequence in the target nucleic acid, i.e. the probe is oligonucleotide specific for a certain target nucleic acid, and is able to hybridize with its target when the target is in a single-stranded form, for example after denaturation. A probe typically comprises 10 to 40 bases, but can also be shorter or longer depending on the application in which it is used.

The term “single-stranded oligonucleotide probe” as used herein means a probe containing one oligonucleotide strand and having a nucleotide sequence that allows hybridization with a target nucleic acid sequence (sense or antisense strand). The term “double-stranded oligonucleotide probe” as used herein means a probe containing two oligonucleotide strands that are at least partially complementary and thus able to hybridize with each other at least in part, one or both having a nucleotide sequence that allows hybridization with a target nucleic acid sequence (sense and/or antisense strand). Typical probes according to the present invention are referred to as double-stranded oligonucleotide probes.

In the context of the current invention, the term “complementarity” shall be understood as a property of double-stranded nucleic acids, such as DNA oligonucleotides, where each of the two strands is complementary to the other in that the base pairs between them are non-covalently connected via two or three hydrogen bonds when in a hybridized (double-stranded) form. If the sequence of one strand, typically referred to as “sense” strand, is known, one can thus reconstruct a complementary strand, typically referred to as “antisense” strand, for such known strand. This is essential for example for nucleic acid amplification using primers and for nucleic acid detection and/or quantification using probes. The term “complementary” as used in the context of the present invention thus refers to the property of an antisense sequence of a nucleic acid to be able to hybridize with the corresponding sense sequence as incorporated in nucleic acid strands. The term “complement” as used herein thus refers to an antisense sequence of a given sense sequence. It is essential in this context to understand that the two strands of a double-stranded nucleic acids run in opposite directions to each other. Thus the complement of a sequence such as “TATGACCA” is written “TGGTCATA”, i.e., the last counterpart bases of a sense strand are considered as the first counterpart bases of an antisense (i.e. complement) strand. The term “mismatch” shall be understood as a pair of nucleotides that is unable to connect via hydrogen bonds to form a base pair in an otherwise complementary double-stranded nucleic acid.

The term “identical” as used in the context of nucleic acid sequences means sequences that are composed of the same bases in the same order, i.e., the sequences are the same. The term refers to a nucleic acid sequence of a certain length, meaning that oligo- or polynucleotides containing a section of identical sequence can also contain additional, non-identical sequence and therefore be distinguishable from one another by characteristics other than the segment of identical sequence.

The terms “essentially complementary” or “essentially identical” shall be understood to cover sequences with at least 70%, preferably at least 80%, more preferably at least 90%, and most preferably at least 95% complementarity or similarity, respectively, with the sequence or sequences to which they are compared to. Term “essential” as used in other contexts in the current invention shall mean “at least 70%”.

The term “partially complementary” as used in the context of the current invention means a pair of oligonucleotides that are able to hybridize with each other only in part, i.e., that cannot exist in a double-stranded format along the entire length of either of the oligonucleotides. Specifically this means that both oligonucleotides contain additional nucleotides the counterparts of which are not present in the other oligonucleotide. These additional nucleotides can, however, be complementary with another oligonucleotide or the sense or antisense strand of a target nucleic acid. The term “full sequence” shall be understood as the entire sequence of an oligonucleotide, which is independent of the length of sequences able to bind to any counterpart.

The terms “target” and “target nucleic acid” refer to the genetic material or a section of it the presence and/or quantity of which in a sample is studied. The term “target nucleotide sequence” refers to a specific section of nucleotide sequence within the target nucleic acid. In the context of the current invention, the target nucleotide sequence can be any region of contiguous nucleotides which is amenable to hybridization with primer and/or probe oligonucleotides.

In order to allow hybridization between oligonucleotides such as primers and/or probes with their respective targets, the oligo- and polynucleotides in the reaction must first be brought in a single-stranded form. The denaturation, or “melting”, of double-stranded oligo- or polynucleotides is the process in which the hybridized strands unwind and separate into single strands through the breaking of hydrogen bonding between the bases of the two strands. Denaturation is achieved by changing the external and/or internal conditions of the reaction in such a way that affects the state of the nucleic acids in the reaction and allows them to become single-stranded. Denaturation is typically achieved by heating the reaction to a temperature above the melting temperature of nucleic acids in the reaction, although the separation of hybridized strands can also be induced e.g. by changing the chemical conditions of the reaction by incorporating chemicals such as urea or sodium hydroxide. The term “melting temperature” (Tₘ) as used herein means the temperature at which 50% of the strands of a specific hybrid are in a double-stranded, i.e. hybridized, form. The Tₘ depends on both the length of the hybrid and the specific nucleotide sequence composition of the hybrid. Hybridization of the nucleic acids in the reaction can be rapidly achieved by using a different set of conditions. Hybridization is typically achieved by cooling the reaction to a temperature at or below the melting temperature of the nucleic acid(s) of interested, in the presence of suitable chemical conditions (e.g. suitable salt concentration and pH). Many sets of conditions suitable for denaturation and hybridization of nucleic acids exist and are known to a person skilled in the art.

Typical alterable conditions of the method of the present invention include, but are not limited to, the temperature where the reaction is allowed to take place, the chemical composition of the reaction, and the time the reaction is allowed to proceed. For example, achieving the denatured state may be achieved using a “set of first conditions” where, for example, the reaction temperature is increased to a level higher than the Tₘ of nucleic acid(s) of interest for a certain period of time. “A set of second conditions” may then be applied, where the reaction temperature is decreased to a level near or below the Tₘ of the nucleic acid(s) of interested for a certain period of time, at a suitable salt concentration and pH.

The term “homogeneous” method, as used herein, refers to a separation-free nucleic acid assay method in which the detection of signal such as emitted fluorescence can be carried out without any physical separation steps such as washing or chromatography.

To detect hybridization of the probe to its target nucleotide sequence, the probe is typically labeled with a molecular marker often referred to as a label. The term “label” as used herein refers to a chemical moiety that is covalently or non-covalently conjugated to an oligonucleotide probe with the purpose of giving the probe a detectable characteristic. Examples of such labels include but are not limited to enzymes, (e.g. alkaline phosphatase and horseradish peroxidase) and enzyme substrates, fluorophores, lanthanide labels (e.g. lanthanide chelates and lanthanide cryptates), chromophores, chemiluminescent labels, electrochemiluminescent labels, ligands having specific binding partners or any other labels.

The term “fluorophore” as used herein refers to a label that emits light upon excitation with light. Examples of suitable fluorophores include but are not limited to prompt fluorophores such as 6-carboxyfluorescein (FAM), tetramethylrhodamine, TAMRA, HEX, TET, JOE, VIC, EDANS and ROX, green fluorescent protein and other fluorescent proteins, fluorescent nucleotides and nucleotide derivatives and analogues; labels with long emission lifetimes such as lanthanide chelates and lanthanide cryptates, preferably europium, terbium, samarium or dysprosium chelates and cryptates; and luminescent particles including but not limited to luminescent particles having a diameter of less than 10 μm.

The term “signal” shall be understood as the measurable and/or quantifiable output generated by a label. Signal can mean any type of measurable signal, including but not being limited to optically measurable signal such as emitted, absorbed or reflected light, or electrochemically measurable signal such as current, voltage or radiation. The methods that allow measuring signal include but are not limited to fluorometry, time-resolved fluorometry, anti-Stokes fluorometry, chemiluminescence, electrochemiluminescence, bioluminescence, phosphorescence, surface Plasmon resonance, fluorescence polarization, absorbance, amperometry, potentiometry, conductometry, and impedimetry. The term “intensity of the signal” means the quantitative strength of the generated and/or detected signal.

The term “quencher” as used herein means a chemical moiety that is covalently or non-covalently conjugated to a probe with the purpose of preventing or quenching the signal emitted by the label. Quenching can occur e.g. by light absorption, in which case the light emitted by the label moiety is absorbed by the quencher moiety, or by some other mechanism which does not necessarily involve spectral absorption. A quencher molecule is typically capable of interacting with the label moiety only when the two molecules are brought in close proximity with each other. Suitable forms of interaction include but are not limited to fluorescence quenching. Examples of suitable quenchers include but are not limited to the dark quenchers Dabcyl, Black Hole Quenchers and QSY7. The term “acceptor” as used herein means a chemical moiety that is covalently or non-covalently conjugated to a probe with the purpose of absorbing the signal emitted by the label and typically but not necessarily transforming it to a different detectable signal. The acceptor can also be a non-overlapping acceptor which does not require spectral absorption. Suitable forms of interaction include but are not limited to fluorescence resonance energy transfer (FRET) and non-overlapping FRET.

The distance of chemical moieties that are capable of interacting with each other when brought in close proximity by the formation of a double-stranded oligonucleotide probe can be expressed as a number of “base pairs” separating the moieties: when the moieties are attached to the same base pair, i.e. to opposite nucleotides in a double-stranded oligonucleotide probe, the difference between the moieties is said to be 0 base pairs. If one of the moieties is relocated by one base in either direction, i.e. attached to a nucleotide one base earlier or one base further in the oligonucleotide sequence, while the other moiety remains attached to the same nucleotide as earlier, the distance between the moieties is said to be 1 base pair. However, it is important to notice that not all nucleotides in a double-stranded oligonucleotide of the present invention have a counterpart in the respective strand. In such a case, the distance of the chemical moieties is calculated as if the counterparting nucleotides where present in the respective strand, even though they are not. The maximal distance between chemical moieties in a double-stranded oligonucleotide probe, as expressed in base pairs, may thus be longer than the length of the complementary (i.e. double-stranded) sequence of said probe.

### Preferred Embodiments of the Invention

There are several designs of competitive hybridization probes known in the art that allow sensitive detection of target nucleic acids in a homogeneous reaction. Examples of such designs can be found in the literature, e.g. in EP 1 339 732, U.S. Pat. No. 5,928,862, EP 0 861 906, EP 1 911 852, Li et al., 2002 and Cheng et al., 2004. Compared to the previously published methods, the current invention allows a clear increase in the detection sensitivity of the target nucleic acid in a nucleic acid assay. This is achieved by enhancing the binding strength of the target nucleic acid to the probes employed, thus allowing competitive advantage of the formation of target-probe hybrids over the formation of double-stranded probe hybrids. The method is based on introducing single-stranded segments in the terminus of both the label and the Quencher probes to increase the Tₘ, and thus the thermostability of the probe-target complexes, as compared with the Tₘ of the double-stranded oligonucleotide probe. The cross-hybridization between the probes and the target nucleic acid will thus dominate over formation of the double-stranded probe, allowing increased detection sensitivity especially of low-copy number target nucleic acids.

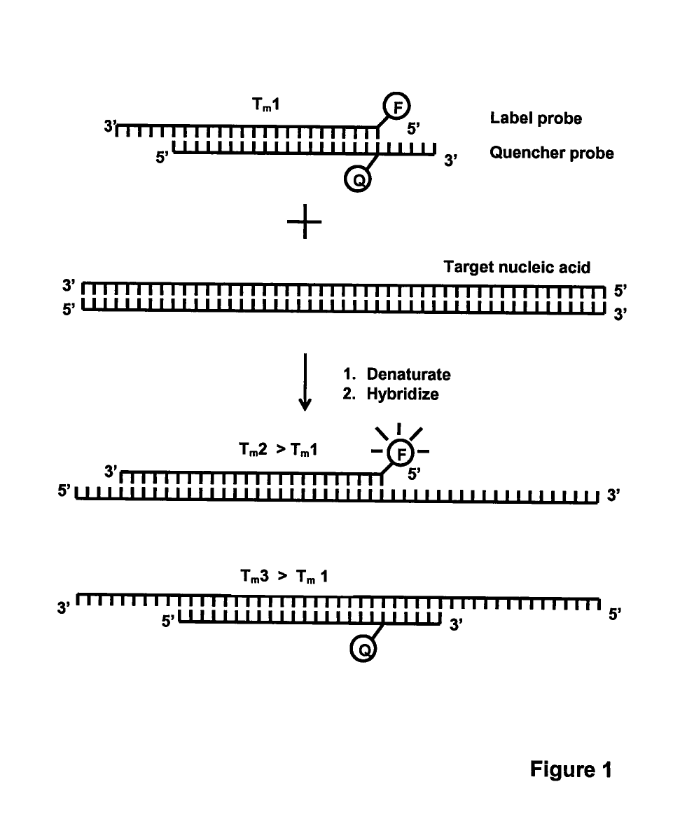

The present invention employs a double-stranded oligonucleotide probe that is composed of two partially complementary oligonucleotides, both oligonucleotides containing additional nucleotides complementary with the double-stranded target nucleic acid only as demonstrated in FIG. 1. One of the probe strands (Label probe) is labelled e.g. with a fluorophore and the other (Quencher probe) e.g. with a quencher. The double-stranded oligonucleotide probe is virtually non-fluorescent due to the close proximity of the fluorophore and the quencher moiety. If a target nucleic acid is present, and all double-stranded oligonucleotides present in the reaction are denatured and then allowed to hybridize, both strands of the probe pair preferentially bind to the complementary strands of the target nucleic acid rather than the complementary probe because of the higher binding strength as defined by the respective Tₘ values. In other words, the higher number of complementary bases between the probe and target strands compared to the number of complementary bases between the two probe strands allows both the label and the Quencher probes to form a thermodynamically more stable duplex with the respective target stand. Any Label probe not hybridized with the Quencher probe emits fluorescence. In the absence of the target nucleic acid, the two probes hybridize with each other to form a non-fluorescent double-stranded oligonucleotide probe.

FIG. 1 shows a schematic drawing of an example of the working principle of a partially complementary double-stranded oligonucleotide probe in the detection of double-stranded target nucleic acid. Both the Label and the Quencher probes have additional bases in either their 5′- or 3′-ends that have no counterparts in the other probe. The additional bases can, however, hybridize with a target nucleic acid, allowing higher Tₘ values for the probe-target duplexes (Tₘ2: Label probe-target hybrid; Tₘ3: Quencher probe-target hybrid) compared to the Tₘ of the Label probe—Quencher probe hybrid (Tₘ1). The interaction between the double-stranded oligonucleotide probe and the double-stranded target nucleic acid can be achieved by first denaturing all nucleic acids in the mixture e.g. by exposing them to a high temperature, after which they are allowed to hybridize with each other e.g. by lowering the temperature of the reaction. When the two probes are hybridized with each other forming a double-stranded oligonucleotide probe, the fluorescence of the label moiety (as indicated by the letter “F”) of the Label probe is substantially quenched by the close proximity of the quencher moiety (indicated by the letter “Q”) of the Quencher probe. When bound to the target present in the reaction, the Label probe becomes substantially unquenched, in this case leading to an increased level of fluorescence. Also the Quencher probe becomes bound to the respective strand of the target nucleic acid.

Contrary to previously published methods, neither of the probes needs to be assigned shorter or longer than the other to ensure maximal performance; in the current method the length difference between the probes becomes irrelevant. Rather, the design of the probes can be decided on a thermodynamic basis, by selecting optimal binding strengths of the probes towards their targets as determined by their Tₘ values. Designing the probes is thus also simplified because the Tₘ can be used as a simple tool to establish suitable probe pairs for any assay setup without extensive experimental assay optimization. For example, for a post-PCR end-point method such as described in WO 2008/093002, optimal Tₘ:s for the probe-target hybrids lie near or below the Tₘ:s of the primers employed to prevent the probes from interfering the PCR and to prevent elongation or cleavage of the probes by the action of the polymerase, also given that the concentration of the probes is kept below that of the primers. An optimal Tₘ for the formation of the double-stranded oligonucleotide probe is approximately 5-15° C. lower than that of the probe-target hybrids, allowing a clear competitive benefit for the latter. The Tₘ of the probes can also be designed so that the probes undergo annealing (hybridization) during the PCR annealing step to allow monitoring of the accumulation of target nucleic acid in real-time, but at the same time allowing the probes to dissociate before the potential extension step at a higher temperature; in this case the optimal Tₘ of the probe-target hybrid is approximately 5-10° C. above the Tₘ of the primers. Alternatively, the Tₘ:s can be designed high enough for the probes to remain hybridized with the target also during the extension step. This makes it is possible to make use of the 3′→5′ exonuclease activity of many polymerases, allowing permanent separation of the label and quencher moieties from the proximity of each other by the irreversible cleavage of the probes, as described e.g. in EP 0 543 942, U.S. Pat. No. 5,538,848 and U.S. Pat. No. 5,804,375. In this case the optimal Tₘ:s for the probe-target hybrid lie approximately 10-15° C. above the Tₘ:s of the primers. In all cases, an approximately 3-30° C., preferably 5-20° C. lower Tₘ compared to the probe-target hybrids can be employed for the double-stranded oligonucleotide probe to favour the formation of the former. In all cases the sensitivity of detection is further enhanced by the effective depletion of the Quencher probe from the reaction, either via binding to the target at the time of measurement, or via irreversible cleavage during the extension step of PCR.

Examples of suitable nucleic acid probes of the present invention may comprise DNA, RNA, non-natural nucleotides, non-natural nucleotide linkages and mixtures of these. The 3′ ends of the probes may be blocked to prevent extension if required, e.g. in assays making use of the 3′→5′ exonuclease activity of the polymerase. Especially in end-point detection methods the probes can be designed short enough not to bind to their targets during amplification and thus not to participate in the amplification reaction.

In the current invention, it is not necessary to determine specific conjugation sites for the label and quencher moieties in the probes, but the label and quencher molecules can be introduced either in internal bases, available e.g. from Thermo Scientific Ulm, Germany, or attached in the terminus of the probes. Similarly to other methods based on competitive hybridization, the method of the current invention can be applied in assays containing either single- or double-stranded target nucleic acids, although the latter are more common and also allow better exploitation of the sensitivity-enhancing characteristics of the current invention.

In a preferred embodiment of the present invention, at least one of the label or quencher moieties is attached to a non-terminal nucleotide of the single-stranded oligonucleotide probe. In another preferred embodiment of the present invention, at least one of the label or quencher moieties is attached to a nucleotide within the complementary sequence of the double-stranded probe. In yet another preferred embodiment of the present invention, all of the label and quencher moieties are attached to nucleotides within the complementary sequence of the double-stranded probe.

The labels to be used in accordance of the present invention include any suitable label known in the art.

In a preferred embodiment of the present invention, the signal detection is based on detecting the fluorescence of an intrinsically fluorescent fluorophore.

As a quencher molecule any suitable molecule capable of interacting with the label moiety when brought in close proximity with each other, e.g. by absorbing the energy of the label moiety can be employed. Suitable forms of interaction include but are not limited to fluorescence resonance energy transfer (FRET) and fluorescence quenching. Examples of suitable light absorbing groups include but are not limited to the dark quenchers Dabcyl, Black Hole Quenchers and QSY7.

In embodiments of the present invention, the label and quencher moieties are attached to the oligonucleotide probes in a manner where the distance between said first and said second label moieties of said double-stranded oligonucleotide probe is not more than 7 base pairs, preferably not more than 4 base pairs, more preferably not more than 2 base pairs and most preferably 0 base pairs, i.e. the label and quencher moieties are attached to exactly opposite nucleotides of the Label and Quencher probes to accomplish maximal proximity and thus efficiency of interaction between the label and quencher moieties of the double-stranded oligonucleotide probe.

The present invention can be used in conjunction with any nucleic acid amplification method, including but not being limited to PCR, ligase chain reaction (LCR), loop-mediated amplification (LAMP), helicase-dependent amplification (HDA), nicking enzyme amplification reaction (NEAR), transcription-mediated amplification (TMA), nucleic acid sequence based amplification (NASBA), and strand displacement amplification (SDA). The present invention can also be used in conjunction with any hybridization assay method without amplification of the target nucleic acid.

The present invention also provides an oligonucleotide sequence that can be employed to specifically amplify and/or detect a nucleic acid sequence specific for Chlamydia trachomatis (C. trachomatis) and a complement thereof. SEQ ID NO: 1 is a 102 bp long fragment of one of the DNA strands of the double-stranded cryptic plasmid. The sequences of the forward and reverse primers employed to amplify the fragment in question are preferably chosen so that they are at least 70% identical to a fragment of the nucleotide sequence presented (SEQ ID NO: 1) and complement thereof, such applicable fragments comprising at least 10 bases, preferably 15 bases and most preferably from 20 to 30. The sequences of the selected primers therefore need not necessarily be completely identical to the sequence in SEQ ID NO: 1 or its complement, i.e. mismatches are admissible provided that the specificity of the amplification is retained. Preferably, the sequences of the primers agree by at least 80% and more preferably by at least 90% with the sequence in SEQ ID No. 1 and said fragments and complements thereof. The primer pair can be designed with the aid of methods and computer programs that are known to the person skilled in the art, e.g. Applied Biosystems Primer Express® Software Version 3.0 which was employed in the current work, taking the applicable PCR conditions into account. Most preferably, the specific forward and reverse primer sequences employed are 5′-CGGCGTCGTATCAAA-GATATGGAC-3′ (SEQ ID NO: 2), and 5′-GAGGAAAACCGTATGAGAAACGGA-TC-3′ (SEQ ID NO: 3), respectively. Additional primer pairs can be employed to amplify a separate sequence of the same or another organism in the same amplification reaction.

The resulting C. trachomatis-specific amplicon can be specifically detected by any probe-using or non-probe-using method known by a person skilled in the art.

Preferably, a partially complementary probe pair designed as provided in the present invention is employed. When a probe or a number of probes are employed, the sequence(s) of the probe(s) is/are preferably chosen so that they are at least 70% complementary to a fragment of the nucleotide sequence presented (SEQ ID NO: 1) and complement thereof, such applicable fragment comprising at least 10 bases, more preferably at least 15 bases, and most preferably between 20 and 30 bases. Preferably, the sequence(s) of the probe(s) agree(s) by at least 80% and most preferably by at least 90% with the sequence in SEQ ID No. 1 and said fragments and complements thereof. Most preferably, a Label probe and a Quencher probe with sequences of 5′-TGATAAAGCA-TCATGCAACATTAACCC-3′ (SEQ ID NO: 4) and 5′-TGTTGCATGATGCTT-TATCTAATGAC-3′ (SEQ ID NO: 5), respectively, are employed.

The present invention also pertains to reagents, compositions, kits, reagents and instruments for use in the detection of target nucleic acids by the methods of the invention. The reagents comprise the competitive probes and possible nucleic acid amplification reagents. Furthermore the kit may comprise the reaction vessel for the nucleic acid amplification reaction. The kit can also comprise the competitive probes and the nucleic acid amplification reagents in dry form in the reaction vessel, such as described in EP 1 766 055. The reagents may also contain any reagents described herein in context of the method of the present invention.

A typical embodiment of the present invention involves a method for detecting nucleic acids wherein a double-stranded oligonucleotide probe comprising
i) a first single-stranded oligonucleotide probe comprising at least one first label moiety capable of emitting a measurable signal, and
ii) a second single-stranded oligonucleotide probe being partially complementary, i.e. an essential part of the probe being essentially complementary, with the first single-stranded oligonucleotide probe and comprising at least one second label moiety capable of interacting with said first label moiety when brought in close proximity with each other, the second label moiety being a quencher or acceptor of emission of the first label moiety;
wherein said first or second probe comprises a sequence being essentially complementary to that of a target nucleotide sequence, and said second or first probe, respectively, comprises a sequence being essentially complementary to a complement of said target nucleotide sequence of said nucleic acid to be detected; and
wherein said first and said second label moieties are attached to said first and second oligonucleotide probes respectively in a manner wherein the distance between said first and second label moieties of said double-stranded oligonucleotide probe is not more than 7 base pairs, preferably not more than 4 base pairs, more preferably not more than 2 base pairs apart and most preferably said first and second label moieties are attached to the same base pair of the said double stranded oligonucleotide probe;

is employed. The method is characteristic in that

a) the complementary sequences of said double-stranded oligonucleotide probe, i.e. the sequences of the first and second probe being essentially complementary to each other, being shorter than the full sequence of either of said first and second single-stranded probes;

b) said first and second probes having a higher Tₘ when hybridized with said target nucleotide sequence compared to the Tₘ of said double-stranded oligonucleotide probe; and

c) the intensity of the signal of said first label when said first oligonucleotide probe is not hybridized to said second probe, i.e. when said first probe is hybridized to the target nucleotide or said second probe is hybridized to the target nucleotide resulting in said first probe not being hybridized to said second probe being higher or lower, preferentially higher, than the intensity of the signal of said first label when said first probe is hybridized to said second probe.

In preferred embodiments of the method of the invention sequences of the first and the second probes not included in the complementary sequences of the double-stranded oligonucleotide probe are essentially complementary to the corresponding sequences of the target nucleic acids. Accordingly in many preferred embodiments the double-stranded oligonucleotide probe does not contain surplus nucleotide sequence but only comprises nucleotide sequence that is essentially complementary to the target nucleic acid or its complement.

It is to be understood that essentially complementary, whenever referred to in the context of the present invention, refers, independent of other referrals, to at least 70% complementarity, preferably at least 80% complementarity and more preferably at least 90% complementarity and most preferably about 100% complementarity.

In many preferred embodiments the first and/or second probes have a 3-30° C., preferably 5-20° C. higher Tₘ when hybridized with the target nucleotide sequence compared to the Tₘ of said double-stranded oligonucleotide probe.

In some preferred embodiments more than one first label moiety and/or second label moiety is comprised in the double-stranded oligonucleotide probe. In some embodiments a first label can interact with more than one second label or vice versa.

In many preferred embodiments the first label moiety is a fluorescent label and the second label moiety is either a fluorescence quencher or a fluorescence acceptor.

In some preferred embodiments at least one of the first or the second label moieties is attached to a non-terminal nucleotide of said first or said second single-stranded oligonucleotide probe.

In many preferred embodiments at least one, preferably at least two, of the first or the second label moieties is attached to a nucleotide within the complementary sequence of said double-stranded probe.

In complex preferred embodiments more than one target nucleic acids, every target nucleic acid having its own double-stranded oligonucleotide probe, are detected in the same reaction.

Preferred methods according to the invention comprise the following steps:

a) providing a mixture of
i) a sample potentially containing the target nucleic acid or acids, and
ii) the double-stranded oligonucleotide probe or probes,

wherein the first and/or second oligonucleotide probe or probes comprise a sequence or sequences being essentially complementary to that of said target nucleotide sequence or sequences, and/or complement or complements thereof;

b) exposing said mixture to conditions wherein said target nucleic acid or acids and said first and said second oligonucleotide probes can assume thermodynamically favoured complexes, by denaturing said nucleic acids present in said mixture resulting in said nucleic acids being in a denatured form using a set of first conditions, and allowing said nucleic acids to hybridize resulting in said nucleic acids being in a hybridized form using a set of second conditions;

c) measuring the signal of said first and/or said second label or labels at least once when said first and said second oligonucleotide probes have assumed said thermodynamically favoured complexes in step b), preferably also measuring at least once said signal of said first and/or said second label or labels, respectively, when said first and said second oligonucleotide probes are in said denatured form in step b);

d) determining the presence, absence or amount of said target nucleic acid in said mixture based on said signal or signals measured in step c).

In especially preferred embodiments of the invention

i) the target nucleic acid to be detected is a product, or

ii) the target nucleic acids to be detected are products of a nucleic acid amplification assay.

In some preferred embodiments of the invention at least one single-stranded oligonucleotide consisting of 10 to 50, preferably 15 to 40, and most preferably 20 to 30 nucleotides, the sequence of said oligonucleotide having at least 70% identity, preferably at least 80% identity, more preferably at least 90% identity and most preferably at least 95% identity to that of an oligonucleotide of equal length selected within SEQ ID NO: 1 or complement thereof, preferably selected from the group consisting of SEQ ID NOS: 2 to 5, 13 and complements thereof, is employed.

Some embodiments of the present invention involve single-stranded oligonucleotides consisting of 10 to 50, preferably 15 to 40, and most preferably 20 to 30 nucleotides. In some of these preferred embodiments the selected oligonucleotides sequences are essentially identical to those of oligonucleotides of essentially equal length selected within SEQ ID NO: 1 and complement thereof. In such embodiments SEQ ID NOS. 2 to 5 and 13 represent preferred alternatives of the invention.

It is to be understood that in the context of the present invention essentially identical, whenever referred to, refers, independent of other referrals, to at least 70% identity, preferably at least 80% identity and more preferably at least 90% identity and most preferably about 100% identity.

In some preferred embodiments of the present invention the sequence of the single-stranded oligonucleotide of the invention selected within SEQ ID NO: 1 and complement thereof is essentially identical to an oligonucleotide of essentially equal length selected within bases 15 to 90, preferably 30 to 75 and most preferably 35 to 70 of SEQ ID NO: 1 and complement thereof. In such embodiments SEQ ID NOS. 4, 5 and 13 represent preferred alternatives of the invention.

In some other preferred embodiments of the present invention the sequence of the single-stranded oligonucleotide of the invention selected within in SEQ ID NO: 1 and complement thereof is essentially identical to an oligonucleotide of essentially equal length selected within the first 50, preferably first 40 and most preferably first 30 bases of SEQ ID NO: 1 and complement thereof. In such embodiments SEQ ID NOS. 2 and 3 represent preferred alternatives of the invention.

Preferred double-stranded oligonucleotides of the invention comprise two single-stranded oligonucleotides as defined above and said sequences of said two single-stranded oligonucleotides being partially complementary with each other.

The present invention also involves use of the above defined oligonucleotides selected within bases 15 to 90, preferably 30 to 75 and most preferably 35 to 65 of SEQ ID NO: 1 and complement thereof in a probe or probes in a nucleic acid amplification method for determining Chlamydia trachomatis.

The present invention further involves use of the above defined oligonucleotide of selected within the first 50, preferably first 40 and most preferably first 30 bases of SEQ ID NO: 1 and complement thereof in a forward or reverse primer, respectively, in a nucleic acid amplification method for determining Chlamydia trachomatis.

### EXAMPLES

The following examples are given to illustrate the invention and should not be read to limit the scope of the invention as claimed in any fashion.

### Example 1

Detection of Neisseria gonorrhoeae

This example illustrates the detection of Neisseria gonorrhoeae (N. gonorrhoeae) using four different designs of competitive probes in homogeneous PCR: 1) equal-length complementary probes (e.g. EP 1 339 732); 2) unequal length complementary probes (e.g. EP 0 861 906); 3-4) partially complementary probes with slightly different Tₘ values (current invention).

To demonstrate the functionality of the present invention, a qualitative PCR assay for N. gonorrhoeae was established. The Label probe (5′-CGTGAAAGTAGCAGG-CGTATAG-3′; SEQ ID NO: 6) was labelled at the 5′ -terminus with an intrinsically fluorescent terbium chelate described in WO 2008/020113. The Quencher probes were labelled with Dabcyl, which is a dark quencher capable of quenching terbium fluorescence when brought in close proximity. Dabcyl was attached at the 3′-terminus of the equal-length (5′-CTATACGCCTGCTACTTTCACG-3′; SEQ ID NO: 7) and unequal length (5′-GCCTGCTACTTTCACG-3′; SEQ ID NO: 8) complementary Quencher probes. For the partially complementary Quencher probes of the present invention with either a 2-bp (5′-GCCTGCTACTTTCACGCT-3′; SEQ ID NO: 9) or a 3-bp (5′-GCCTGCTACTTTCACGCTG-3′; SEQ ID NO: 10) single-strand extension, Dabcyl was introduced internally via a modified thymidine base (position in sequence shown in bold).

The assay setup was based on the dry chemistry principle described in EP 1 766 055. When rehydrated with 30 μL of sample, the reaction contained 15 nM of Label probe, 150 nM of Quencher probe and 500 nM of the forward (5′-CCGGAACTGGTTTCATCTGATT-3′; SEQ ID NO: 11) and reverse (5′-GTT-TCAGCGGCAGCATTCA-3; SEQ ID NO: 12) primers, along with other generic components required for PCR. The reactions were carried out in a prototype GenomEra nucleic acid analyzer (Abacus Diagnostica) that uses the technical solutions described in EP 1 771 250. After initial denaturation at 100° C. for 115 s, 45 cycles of amplification were carried out, consisting of 27° C. for 1.7 s, 62° C. for 16 s, 108° C. for 7.2 s, and 100° C. for 4.5 s. The temperatures given refer to the settings of the heat blocks, not to the temperatures inside the reaction vessels.

After the PCR reactions were completed, the fluorescence was automatically measured in a time-resolved manner within the same instrument. The method of the end-point detection was based on a two-step detection principle described in WO 2008/093002. In short, time-resolved fluoresce signal of terbium was measured twice, first when all or nearly all oligonucleotides including the probes were in a denatured form (yielding maximum intensity of fluorescence) and again when all or nearly all oligonucleotides including the probes were in a hybridized from (only the unqueched portion of the Label probe emitting fluorescence), the ratio of giving the percentage of unquenched Label probe in that specific reaction. The signal-to-noise (S/N) values blotted in the graph were calculated by comparing the ratios of the positive reactions to the ratios of negative control reactions.

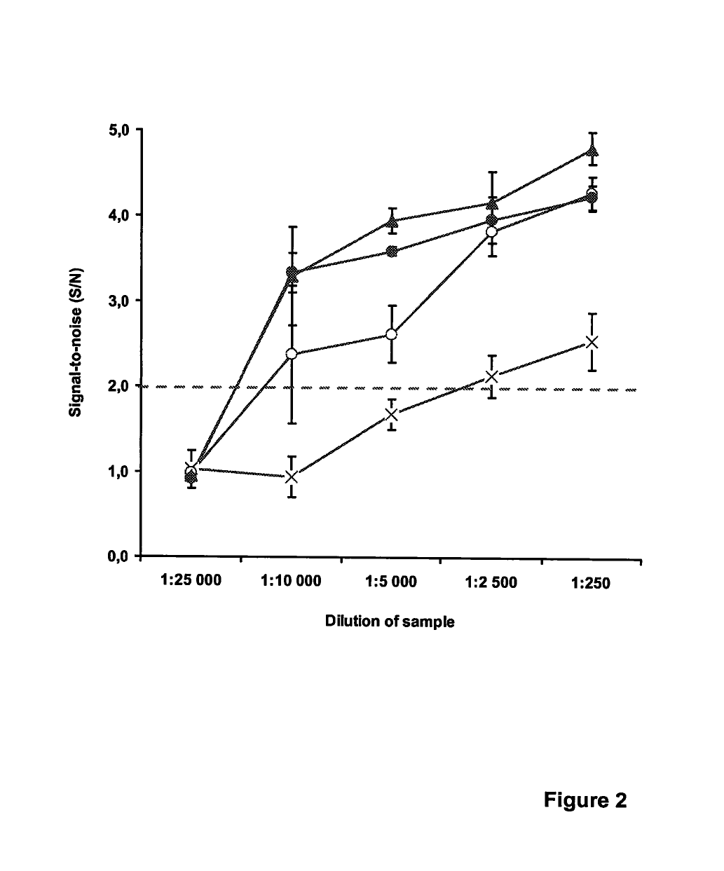

The results of the experiment are shown in FIG. 2. The Tₘ of all the probes studied was kept low to prevent the probes from participating in PCR by any mechanism. The Tₘ:s of the different probes are given in the Table 1. As can be seen in FIG. 2, the sensitivity of detection of low amounts of the target organism in the sample increases when single-stranded segments are added in either the Label probe alone (resulting in an increase of detection sensitivity with, however, two of the four 1:10 000 diluted replicates still remaining negative) or in both the label and Quencher probes as presented in the current invention (resulting in a significant increase of detection sensitivity). The experiment thus demonstrates that the present invention allows more sensitive detection of a target nucleic acid by a mechanism where also the Quencher probe forms thermodynamically more stable complexes with the target nucleic acid compared with complexes formed with the Label probe. This was achieved by introducing additional nucleotides in the 3′-terminus of both the Quencher probe and the Label probes, the counterparts of which were not present in the other probe but were present in the target nucleic acid specific for N. gonorrhoeae. Very sensitive detection was achieved with a design using partially complementary probes of the current invention where the Tₘ:s for the Label probe-target and the Quencher probe-target duplexes were approximately the same (−56° C.). Thus, compared with equal-length, complementary probes of approximately the same Tₘ:s (−56° C.), a significantly higher sensitivity of detection could be achieved despite the fact that there was no difference in the thermostability of the probe-target complexes between these two probe designs. However, as can be seen in FIG. 2, very sensitive detection could also be achieved even if there was a difference of approximately 6° C. between the Tₘ:s of the Label probe-target (−56° C.) and the Quencher probe-target (−50° C.) duplexes, indicating that even a relatively short single-stranded extension in the Quencher probe effectively shifts the equilibrium towards binding to the target nucleic acid rather than to the Label probe. This may be valuable e.g. in cases where only a short conserved genome segment is available for assay development, which is the case for e.g. many pathogenic organisms capable of spontaneous mutagenesis.

FIG. 2 illustrates end-point detection of N. gonorrhoeae using four different designs of competitive probes in homogeneous PCR. The detection sensitivity of the different probe designs was studied by sequential dilutions of a N. gonorrhoeae-positive pure-cultured sample in microbiology-grade water. A typical cut-off level is indicated with a dotted line. The values shown are the mean of four replicates, with the standard deviations shown with error bars. The probe designs and their respective Tₘ values and length in basepairs are shown in Table 1: (a) The Tₘ values and the length (bp; basepairs) are given for the complementary sections only. The Tₘ:s were calculated using the Applied Biosystems Primer Express® Software Version 3.0; (b) The internal quencher moiety (Dabcyl-dT; introduced as a modified thymidine base by Thermo Scientific) was located at the site of the nearest possible thymine base to allow maximal proximity to the label moiety of the Label probe, but at the same time avoiding to introduce a mismatch in the Quencher probe sequence. Such a base could be found 4 bases upstream in the 3′→5′ direction of the Quencher probe compared to the original location. In a separate study, it was confirmed that there were no significant changes in the quenching efficiency up to a 7 base difference between the Dabcyl and terbium moieties (data not shown).

TABLE 1
End-point detection of N. gonorrhoeae using four different designs of competitive probes in
homogeneous PCR

| | | Label probe with a 6-bp single-strand |
|---|---|---|
| | | segment |
| | Equal-length, | Unequal-length |
| | complementary | complementary probes |
| | probes (x) | (o) |
| Neisseria gonorrhoeae | 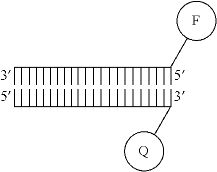 | 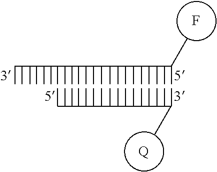 |
| Tₘ1 | 55.7° C./22 bp^{a} | 46.1° C./16 bp |
| (Double-stranded probe) | | |
| Tₘ2 | 55.7° C./22 bp | 55.7° C./22 bp |
| (Label probe + target) | | |
| Tₘ3 | 55.7° C./22 bp | 46.1° C./16 bp |
| (Quencher probe + target) | | |
| | Label probe with a 6-bp single-strand segment | |
| | Partially complementary probes^{b} | |
| | (current invention) | |
| | Quencher probe with a | Quencher probe with a |
| | 2-bp single-strand | 3-bp single-strand |
| | extension (▴) | extension (•) |
| Neisseria gonorrhoeae | 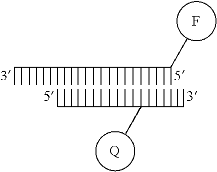 | 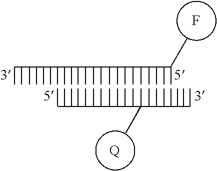 |
| Tₘ1 | 46.1° C./16 bp | 46.1° C./16 bp |
| (Double-stranded probe) | | |
| Tₘ2 | 55.7° C./22 bp | 55.7° C./22 bp |
| (Label probe + target) | | |
| Tₘ3 | 50.0° C./18 bp | 55.9° C./19 bp |
| (Quencher probe + target) | | |

### Example 2

Detection of Chlamydia trachomatis

This example illustrates the detection of Chlamydia trachomatis using unequal length complementary probes and partially complementary probes in a qualitative homogeneous end-point PCR. In order to further demonstrate that increasing the Tₘ of the Quencher probe-target duplex has as a positive effect on the detection sensitivity, another set of probe pairs were designed that were specific for the cryptic plasmid of C. trachomatis. An assay setup similar to Example 1 was employed, now concentrating on the two main probe design alternatives, i.e., the unequal length complementary probes (e.g., EP 0 861 906) and the partially complementary probes of the current invention. To study the effect of introducing a mismatch in the single-stranded segment of the Quencher probe, the internal quencher moiety (Dabcyl-dT) was positioned at an exactly opposite base of the label moiety on the other probe, resulting in a mid-sequence T-T mismatch (original base A replaced by T) between the Quencher probe and the target nucleic acid. The nucleotide sequence of the Label probe was 5′-TGATAAAGCA-TCATGCAACATTAACCC-3′ (SEQ ID NO: 4), that of the unequal length complementary Quencher probe 5′-GTTGCATGATGCTTTATCA-3′ (SEQ ID NO: 13; 3′-terminal Dabcyl), and that of the partially complementary Quencher probe of the present invention 5′-TGTTGCATGATGCTTTATCTAATGAC-3′ (SEQ ID NO: 5; position of the internal Dabcyl shown in bold). The Label probe was labelled at the 5′-terminus with an intrinsically fluorescent terbium chelate similarly to Example 1. The nucleotide sequences of the forward and reverse primers were 5′-CGGCGTCGTATCAAAGATATGGAC-3′ (SEQ ID NO: 2) and 5′-GAGGAAAACCGTATGAGAAACGGATC-3′ (SEQ IS NO: 3), respectively.

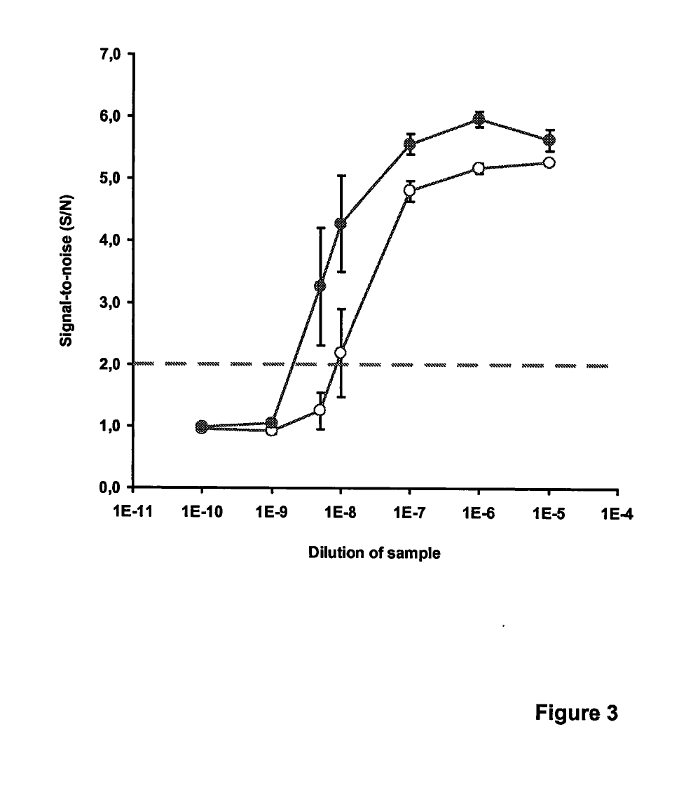

The results of the experiment are shown in FIG. 3 wherein end-point detection of C. trachomatis in homogeneous PCR using unequal length complementary probes (∘) and partially complementary probes of the current invention () is shown. The respective Tₘ-values and length of the probes are shown in Table 2: (a) The Quencher probe contains a single-nucleotide mismatch at the site of the internal quencher moiety (Dabcyl-dT), resulting in a mid-sequence T-T mismatch between the Quencher probe and the target nucleic acid. A typical cut-off level is indicated with a dotted line. The values shown are the mean of four replicates, with the standard deviations shown with error bars. The Tₘ:s of the probes employed are given in the table below the graph.

TABLE 2
End-point detection of C. trachomatis in homogeneous PCR using
unequal length and partially complementary probes

| | Label probe with a 8-bp single-strand segment | |
|---|---|---|
| | Unequal length | Partially complementary |
| | complementary probes (o) | probes (•) |
| Chlamydia trachomatis | 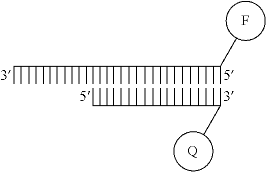 | 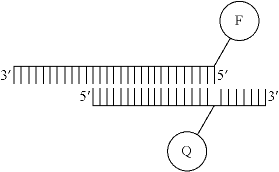 |
| Tₘ1 | 48.9° C./ 19 bp | 48.9° C./19 bp |
| (Double-stranded probe) | | |
| Tₘ2 | 62.8° C./27 bp | 62.8° C./27 bp |
| (Label probe + target) | | |
| Tₘ3 | 48.9° C./19 bp | 58.3° C./26 bp* |
| (Quencher probe + target) | | |

It can be concluded that the detection of the target organism could be rendered more sensitive even despite a single-base mismatch in the Quencher probe-target duplex, based on the fact that the formation of this hybrid was still thermodynamically favoured over the formation of the double-strand probe hybrid and that suitable hybridization conditions were used. This can be made of use at least two ways: firstly, the assay development is made easier as most base modifications including label modifications available for one base only can be directly employed even when strict proximity requirements do not allow locating the label or quencher moiety to the nearest suitable base. The position of the labels or quenchers can thus be more easily adjusted to allow optimal energy transfer or absorbance in all cases. Alternatively, additional internal quencher moieties can be included in the Quencher probe, further increasing the quenching efficiency and thus the background fluorescence of the established assay. The label and the quencher moieties can thus be located on any combination of base pairs as long as the signal of the label is effectively quenched when the probes are in a double-stranded form. It has been demonstrated e.g. in U.S. Pat. No. 5,538,848 that some of the common fluorophores and quenchers can be situated remotely and still be operative.

Furthermore, single-nucleotide mutations are very prevalent also amongst pathogenic organisms. Discrimination of such organisms by a single-nucleotide mutation may not be desirable in cases where all strains need to be captured in one assay and detected with similar sensitivity. Moreover, highly conserved genome segments may not be available for the probe design in the case of many pathogenic organisms, including viruses and bacteria capable of spontaneous mutagenesis. However, it must be noted that while the partially complementary probes of the current invention can be employed to allow sensitive detection of target organisms despite single-nucleotide mutations, this does not decrease the specificity of the assay or increase the fluorescence background signal of the assay because of the effective competition for the Label probe by the Quencher probe, as described in the current and all earlier publications on competitive hybridization, such as in U.S. Pat. No. 5,928,862, EP 1 339 732, EP 0 861 906, Li et al., 2002 and Cheng et al., 2004. The non-mismatched duplex of the label and Quencher probe can easily overcome the formation of any duplexes that are thermodynamically less stable and the probes will thus not bind to alternative targets when the free energy produced is at a lower level compared to that of the double-stranded oligonucleotide probe. The preferred sites of allowing the potential single-nucleotide mutations are at the single-stranded segments of either of the probes. For sensitive detection of single-nucleotide mutations e.g. in the diagnosis of hereditary diseases, however, the Tₘ difference of the different complexes should be optimized more carefully, using more subtle single-stranded extensions on both probes and careful optimization of the hybridization conditions. Surplus nucleotide sequence, i.e. sequence that is not present in the target nucleic acid, should in each case be avoided in the probes to minimize any unintentional hybridization reactions.

### Example 3

Homogenous Real-Time Monitoring of Methicillin-Resistant Staphylococcus aureus

This example illustrates homogeneous real-time monitoring of the accumulation of methicillin-resistant Staphylococcus aureus (MRSA)-specific DNA in homogeneous PCR using unequal-length complementary probes and the partially complementary probes of the current invention. To demonstrate that the present invention allows more sensitive detection of target nucleic acids also in terms of an earlier threshold cycle (Cₜ) in real-time PCR applications, we established an assay for MRSA applying the same basic principles than in the previous experiments. However, the real-time measurement was performed as a single measurement step at 40° C. after every second PCR cycle. Fluorescence recording was started after the 11^{th} PCR cycle. The nucleotide sequence of the Label probe was 5′-AAGGAATAGTGTAGATTACGTTAGACCTT-3′ (SEQ ID NO: 14), that of the unequal-length complementary Quencher probe was 5′-AACGTAATCTACACT-ATTCCTT-3′ (SEQ ID NO: 15; 3′-terminal Dabcyl), and that of the partially complementary Quencher probe of the present invention was 5′-AACGTAATCTACACTATTCCTTCTATAA-3′ (SEQ ID NO: 16; position of the internal Dabcyl shown in bold). The Label probe was labelled at the 5′-terminus with an intrinsically fluorescent terbium chelate similarly to Example 1. The nucleotide sequences of the forward and reverse primers were 5′-GAAAGAGCAATCAAAAATGAAGACATAG-3′ (SEQ ID NO: 17) and 5′-TGGATGTCCTTGGACTGATATATAAGA-3′ (SEQ ID NO: 18), respectively.

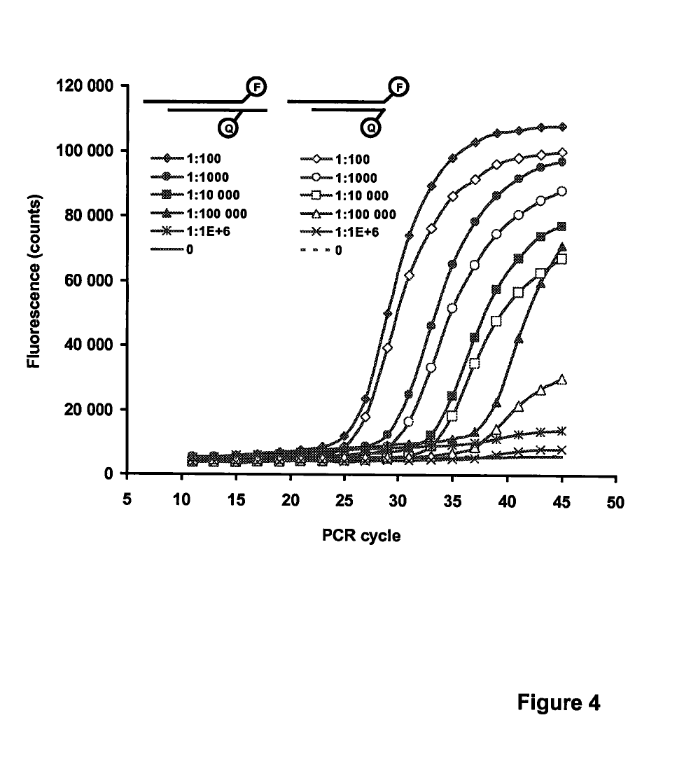

FIG. 4 illustrates homogeneous real-time monitoring of the accumulation of MRSA-specific DNA in PCR using unequal-length complementary probes (open symbols) and partially complementary probes of the current invention (filled symbols). The respective Tₘ-values and length of the probes are shown in Table 3. After initial 10 PCR cycles, measurement was performed after every two PCR cycles at 40° C. The following sample dilutions were analyzed (symbols used in the FIG. 4 shown in parentheses): 1:100 (⋄/♦), 1:1 000 (∘/), 1:10 000 (□/▪), 1:100 000 (Δ/▴) and 1:1 000 000 ( [symbol] /x). Negative reactions are indicated with dotted (unequal-length complementary probes) and solid (partially complementary probes) lines with no symbols. The probes were designed so that the Dabcyl quencher and the terbium chelate could be positioned at opposed bases of the two strands.

TABLE 3
Homogeneous real-time monitoring of the accumulation of MRSA-specific DNA in PCR

| | Label probe with a 7-bp single-strand segment | |
|---|---|---|
| | Unequal-length complementary | Partially complementary probes |
| | probes (open symbols, e.g. o) | (filled symbold, e.g. •) |
| MRSA | 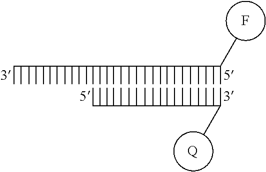 | 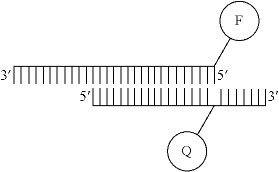 |
| Tₘ1 | 46.2° C./22 bp | 46.2° C./22 bp |
| (Double-stranded probe) | | |
| Tₘ2 | 56.1° C./29 bp | 56.1° C./29 bp |
| (Label probe + target) | | |
| Tₘ3 | 46.2° C./22 bp | 52.1° C./28 bp* |
| (Quencher probe + target) | | |

As can be seen in FIG. 4, at all sample dilutions the partially complementary probe pair of the current invention gave a fluorescent signal distinguishable from the background fluorescence earlier than the corresponding unequal length complementary probe pair. In conclusion, the present invention allows earlier detection of a target nucleic acid in real-time PCR, translating into higher assay sensitivity at any target concentration.

### Other Preferred Embodiments

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

1-11. (canceled)

12. A method for detecting a nucleic acid, comprising
a) providing a mixture of
i) a sample potentially containing the target nucleic acid, and
ii) at least one double-stranded oligonucleotide probe comprising
A) a first single-stranded oligonucleotide probe comprising at least one first label moiety capable of emitting a measurable signal, and
B) a second single-stranded oligonucleotide probe being partially complementary such that an essential part of the probe is essentially complementary with the first single-stranded oligonucleotide probe and further comprising at least one second label moiety capable of interacting with said first label moiety when brought in close proximity with each other, the second label moiety being a quencher or acceptor of emission of the first label moiety;
wherein said first or second oligonucleotide probe comprises a sequence which is essentially complementary to that of a target nucleotide sequence of said nucleic acid, and said second or first oligonucleotide probe, respectively, comprises a sequence which is essentially complementary to a complement of said target nucleotide sequence;
wherein complementary sequences of said double-stranded oligonucleotide probe are shorter than the full sequence of both said first and second single-stranded oligonucleotide probes;
wherein sequences of the first and second oligonucleotide probes not included in the complementary sequences of the double-stranded nucleotide probe are essentially complementary to corresponding sequences of the target nucleic acid;
wherein said first and second oligonucleotide probes have a higher Tₘ when hybridized with said target nucleotide sequence compared to the Tₘ of said double-stranded oligonucleotide probe; and
wherein said first and said second label moieties are attached to said first and second oligonucleotide probes respectively in a manner wherein the distance between said first and second label moieties of said double-stranded oligonucleotide probe is not more than 7 base pairs apart;
b) exposing said mixture to conditions wherein said target nucleic acid and said first and said second oligonucleotide probes can assume thermodynamically favored complexes, by denaturing said nucleic acids present in said mixture resulting in said nucleic acids being in a denatured form using a set of first conditions, and allowing said nucleic acids to react by hybridization, resulting in said nucleic acids being in a hybridized form using a set of second conditions;
c) measuring the signal of said first and/or said second label at least once when said first and said second oligonucleotide probes have assumed said thermodynamically favored complexes in step b), the intensity of the signal of said first label when said first oligonucleotide probe is not hybridized to said second oligonucleotide probe being higher or lower than the intensity of the signal of said first label when said first oligonucleotide probe is hybridized to said second oligonucleotide probe;
d) determining the presence, absence or amount of said target nucleic acid in said mixture based on said signal measured in step c).

13. The method of claim 12, wherein essentially complementary, when referred to, refers, independent of other referrals, to at least 70% complementarity.

14. The method of claim 12, wherein the first and/or second oligonucleotide probes have a 3-30° C. higher Tₘ when hybridized with the target nucleotide sequence compared to the Tₘ of self-hybridized double-stranded oligonucleotide probe.

15. The method of claim 12, wherein said double-stranded oligonucleotide probe comprises more than one first label moiety and/or second label moiety.

16. The method of claim 12, wherein the first label moiety is a fluorescent label and the second label moiety is either a fluorescence quencher or a fluorescence acceptor.

17. The method of claim 12, wherein at least one of the first or the second label moieties is attached to a non-terminal nucleotide of said first or said second single-stranded oligonucleotide probe.

18. The method of claim 12, wherein at least one of the first or the second label moieties is attached to a nucleotide within the complementary sequence of said double-stranded probe.

19. The method of claim 12, wherein multiple target nucleic acids may be present in said sample, and a double-stranded oligonucleotide specific for each target nuclei acid is provided, such that every target nucleic acid having its own double-stranded oligonucleotide probe is detected in a same hybridization reaction step.

20. The method of claim 12, wherein the target nucleic acid to be detected is a product of a nucleic acid amplification assay.

21. The method of claim 20, wherein said first single-stranded oligonucleotide probe comprises at least one single-stranded oligonucleotide consisting of 10 to 50 nucleotides, the sequence of said oligonucleotide having at least 70% identity to that of an oligonucleotide of equal length selected within SEQ ID NO: 1 or complement thereof.


SEQUENCE LISTING (18 sequences)

SEQ ID NO: 1 - DNA, 102 bp, Chlamydia trachomatis
cggcgtcgtatcaaagatatggacaaatcgtatctcgggttaatgttgcatgatgctttatcaaatgacaagcttagatccgtttctcatacggttttcctc

SEQ ID NO: 2 - DNA, 24 bp, Artificial
  Forward primer ex. 2
cggcgtcgtatcaaagatatggac

SEQ ID NO: 3 - DNA, 26 bp, Artificial
  Reverse primer ex. 2
gaggaaaaccgtatgagaaacggatc

SEQ ID NO: 4 - DNA, 27 bp, Artificial
  Label probe ex. 2
tgataaagcatcatgcaacattaaccc

SEQ ID NO: 5 - DNA, 26 bp, Artificial
  Quencher probe ex. 2
tgttgcatgatgctttatctaatgac

SEQ ID NO: 6 - DNA, 22 bp, Artificial
  Label probe ex. 1
cgtgaaagtagcaggcgtatag

SEQ ID NO: 7 - DNA, 22 bp, Artificial
  Quencher probe ex. 1, equal-length
ctatacgcctgctactttcacg

SEQ ID NO: 8 - DNA, 16 bp, Artificial
  Quencher probe ex. 1, unequal length
gcctgctactttcacg

SEQ ID NO: 9 - DNA, 18 bp, Artificial
  Quencher probe ex. 1, part. comp. 2-bp
gcctgctactttcacgct

SEQ ID NO: 10 - DNA, 19 bp, Artificial
  Quencher probe ex. 1, part. comp. 3-bp
gcctgctactttcacgctg

SEQ ID NO: 11 - DNA, 22 bp, Artificial
  Forward primer ex. 1
ccggaactggtttcatctgatt

SEQ ID NO: 12 - DNA, 19 bp, Artificial
  Reverse primer ex. 1
gtttcagcggcagcattca

SEQ ID NO: 13 - DNA, 19 bp, Artificial
  Quencher probe ex. 2, unequal length
gttgcatgatgctttatca

SEQ ID NO: 14 - DNA, 29 bp, Artificial
  Label probe ex. 3
aaggaatagtgtagattacgttagacctt

SEQ ID NO: 15 - DNA, 22 bp, Artificial
  Quencher probe ex. 3, unequal length
aacgtaatctacactattcctt

SEQ ID NO: 16 - DNA, 28 bp, Artificial
  Quencher probe ex. 3, part. comp.
aacgtaatctacactattccttctataa

SEQ ID NO: 17 - DNA, 28 bp, Artificial
  Forward primer ex. 3
gaaagagcaatcaaaaatgaagacatag

SEQ ID NO: 18 - DNA, 27 bp, Artificial
  Reverse primer ex. 3
tggatgtccttggactgatatataaga